(19) United States
(12) Patent Application Publication
(10) Pub. No.: US 2022/0273557 A1
(43) Pub. Date: Sep. 1, 2022

(54) COMPOSITIONS AND METHODS FOR TREATMENT OF PRESBYOPIA

(71) Applicant: Allergan Sales, LLC, Madison, IL (US)

(72) Inventors: Mohammed Dibas, Corona, CA (US); Daniel Gil, Corona Del Mar, CA (US); Arthur Bradley, Bloomington, IN (US); Renfeng Xu, Omaha, NE (US)

(21) Appl. No.: 17/597,769

(22) PCT Filed: Jul. 24, 2020
(86) PCT No.: PCT/US20/43530

§ 371 Date: Jan. 21, 2022

Related U.S. Application Data
(60) Provisional application No. 62/879,285, filed on Jul. 26, 2019.

(51) Int. Cl. A61K 9/00; A61K 31/4985; A61K 47/38; A61K 47/02; A61P 27/10

(52) U.S. Cl. CPC A61K 9/0048; A61K 31/4985; A61K 47/38; A61K 47/02; A61P 27/10

(57) ABSTRACT

The present invention is related to topical ophthalmic compositions comprising brimonidine. Also described herein are methods for the treatment of ocular conditions (e.g., presbyopia), methods for improving near reading speed in a subject with presbyopia, methods for improving near vision in a subject with presbyopia, and methods for reducing pupil diameter in a subject with presbyopia using the topical ophthalmic compositions.

### BACKGROUND OF THE INVENTION

### Field of the Invention

Certain embodiments described herein relate to topical ophthalmic compositions comprising brimonidine and methods of treating presbyopia using such topical ophthalmic compositions.

### Background of the Invention

Ocular pupil size is determined by the opposing forces provided by the iris sphincter and dilator muscles, with the former innervated by parasympathetic nerves using acetylcholine as a neurotransmitter targeting muscarinic receptors, and the latter by sympathetic nerves which release norepinephrine acting on adrenergic receptors.

Small pupils have become a promising alternative treatment for presbyopia due to their ability to expand the depth of focus of the human eye. Reducing pupil size can improve near visual acuity, near reading speed, and near image quality in distance corrected presbyopic eyes. To maximize the near vision gains, very small, e.g. 1-2 mm pupils are ideal, but these very small pupils can compromise distance vision, especially at low light levels. However, at night small pupils can reduce vision complaints of halos and starbursts. At photopic light levels with a focused retinal image, maximum visual acuity is achieved with small pupils (2-3 mm), but as retinal illuminance decreases larger pupils are needed to optimize focused vision. Therefore, employing a fixed small pupil ideal for high light levels, will compromise distance vision at low light environments, and a pupil miosis that is ideal for distance vision at low light levels will provide reduced near vision gain at high light levels.

There is a need in the art for improved topical ophthalmic compositions that produce sufficient pupil miosis to improve near acuity and reading while maintaining distance vision over a range of environmental light levels. The present disclosure addresses this need.

### SUMMARY OF THE INVENTION

Certain embodiments relate to methods of treating presbyopia in a subject in need of treatment thereof. The methods comprise administering to at least one eye of the subject a therapeutically effective amount of a topical ophthalmic composition comprising brimonidine.

Certain embodiments further provide methods for improvement of near vision in a subject with presbyopia in need of treatment thereof. The methods comprise administering to at least one eye of the subject a therapeutically effective amount of a topical ophthalmic composition comprising brimonidine.

Certain embodiments also provide methods of reducing pupil diameter in a subject with presbyopia in need of treatment thereof. The methods comprise administering to at least one eye of the subject a therapeutically effective amount of a topical ophthalmic composition comprising brimonidine.

Certain embodiments further provide methods of improving near reading speed in a subject with presbyopia in need of treatment thereof. The methods comprise administering to at least one eye of the subject a therapeutically effective amount of a topical ophthalmic composition comprising brimonidine.

In some embodiments, there is provided a method of treating presbyopia in a subject in need of treatment thereof, comprising administering to at least one eye of the subject a therapeutically effective amount of a topical ophthalmic composition comprising 0.1% (w/v) brimonidine, 0.5% (w/v) carboxymethyl cellulose, 0.005% (w/v) stabilized oxychloro complex, 0.6% (w/v) boric acid, 0.045% (w/v) sodium borate decahydrate, 0.37% (w/v) sodium chloride, 0.14% (w/v) potassium chloride, 0.006% (w/v) calcium chloride, 0.006% (w/v) magnesium chloride, and NaOH and/or HCl. Additionally, the topical ophthalmic composition has a pH of 7.7.

In some embodiments, there is provided a topical ophthalmic composition for use in the treatment of presbyopia. The topical ophthalmic composition comprises 0.1% (w/v) brimonidine, 0.5% (w/v) carboxymethyl cellulose, 0.005% (w/v) stabilized oxychloro complex, 0.6% (w/v) boric acid, 0.045% (w/v) sodium borate decahydrate, 0.37% (w/v) sodium chloride, 0.14% (w/v) potassium chloride, 0.006% (w/v) calcium chloride, 0.006% (w/v) magnesium chloride, and NaOH and/or HCl. Additionally, the topical ophthalmic composition has a pH of 7.7.

### BRIEF DESCRIPTION OF THE FIGURES

FIG. 1 shows Pupil plane image (404×134 pixels) excerpted from a single video frame. Custom pupilometer software fit red and blue circles, respectively, to both the 6 mm white calibration dots and the pupils.

FIG. 2 shows Pupil diameter in mm plotted as a function of time in hours after early morning brimonidine dosing. Data points represent the mean of 3 repeat measures and 30 subjects (19 subjects <50 y and 11 subjects >50 y). In each panel, pupil diameter for right (filled symbols) and left (open symbols) eyes are plotted as subjects viewed high reflectance materials with the room illuminated with 2000, 200, 20, and 0 lux. Pupil diameters observed when viewing distant (5 m, top panels) and near (40 cm, bottom panels) stimuli are plotted for ages >50 years (left column), ages 40-50 years (right column).

FIG. 3 shows pupil diameter plotted as a function of environmental light level (lux) before (solid) and 1 hour after (dashed) bilateral instillation of a single drop of 0.1% ALPHAGAN P™ (brimonidine tartrate ophthalmic solution). Red and blue symbols represent pupil while viewing distant (5 m) and near (40 cm) stimulus, respectively. Error bars are the standard error of the mean.

FIG. 4 shows log MAR visual acuity plotted as a function of time in hours after bilateral early morning dosing with a single drop of 0.1% ALPHAGAN P™. Visual acuities are plotted as subjects viewed high reflectance materials with the room illuminated at 2000, 200, 20 lux, when binocularly viewing distant (5 m, top panels) and near (40 cm, bottom panels) stimuli. Acuities for subjects >50 years and ages 40-50 years are plotted in the left and right panels, respectively

FIG. 5 illustrates Scatter plots showing change in NEAR log MAR visual acuity produced by brimonidine (i.e., t_{1HR}−t₀) as a function of subject age in years (FIG. 5A) and pupil change in mm (FIG. 5B). Red arrows indicate improvements in visual acuity and reductions in pupil diameter.

FIG. 6 shows percent of subjects experiencing a 0.1 log MAR (A) or 0.2 log MAR (B) improvement in visual acuity 1 hour after dosing with 0.1% ALPHAGAN P™. Percentages for the younger (40-50, blue) and older (>50, red) are plotted for 20, 200 and 2000 lux of room illumination

FIG. 7 shows reading speed (words per minute) at 1 hour after dosing (FIG. 7A) and the improvement in reading speed (in words per minute) due to pupil miosis at 1 hour (FIG. 7B) are plotted as a function of letter size. The red, blue, and purple solid curves represent reading speed (FIG. 7A) or reading speed improvement (FIG. 7B) at 20, 200, and 2000 lux, respectively. The open circles, squares, and triangles in both FIGS. 7A and 7B indicate typical font size seen in newspaper (0.45 log MAR at 40 cm) and cellphone text message (0.45 log MAR at 40 cm), grocery labels (0.38 log MAR at 40 cm), and OTC labels (0.3 log MAR at 40 cm), respectively.

### DETAILED DESCRIPTION OF THE INVENTION

Unless defined otherwise, all technical and scientific terms used herein have the same meaning as commonly understood by one of ordinary skill in the art to which the subject matter pertains.

As used in this specification and the appended claims, the singular forms “a,” “an” and “the” include plural referents unless the context clearly dictates otherwise.

The invention provides topical ophthalmic compositions comprising brimonidine. The invention further provides topical ophthalmic compositions for use in the treatment of presbyopia, wherein the topical ophthalmic compositions comprise brimonidine. The term “topical” as used herein refers to an ophthalmic composition intended for direct application to the corneal surface of an eye of a subject in need thereof. The term topical does not include injections to the eye of a subject (e.g., anterior chamber injections).

The term “ophthalmic composition” or “ophthalmic compositions of the invention” as used herein refers to compositions suitable for application to an eye of a subject, which are in such form as to permit the biological activity of brimonidine to be effective, and which contain no additional components that are unacceptably toxic to the subject to which the composition would be administered. Such ophthalmic compositions will generally be sterile. Thus, for topical application to the eye, the ophthalmic compositions of the present invention will generally be formulated as sterile aqueous compositions (e.g., suspensions, solutions, emulsions or the like) and typically include at least 70 w/v %, more typically 80 w/v % and even more typically at least 90 or 95 w/v % purified water. Such ophthalmic compositions may be in the form of liquid preparations, e.g., eye drops. The ophthalmic compositions may be suitable for single-dose or multiple-dose topical application. The ophthalmic compositions suitable for multi-dose topical application are often disposed in a dispenser (e.g., an eye dropper), which can dispense the ophthalmic composition (e.g., as individual drops) to the corneal surface of the eye.

In certain aspects, the topical ophthalmic compositions of the invention comprise at least about 0.01% w/v brimonidine. In other aspects, the topical ophthalmic compositions of the invention comprise less than about 0.01% w/v brimonidine. In some embodiments, the topical ophthalmic compositions of the invention comprise brimonidine at a concentration from about 0.01% w/v to about 20% w/v. In additional embodiments, the topical ophthalmic compositions of the invention comprise brimonidine at a concentration from about 0.01% w/v to about 2% w/v. In specific embodiments, the topical ophthalmic compositions of the invention comprise brimonidine at a concentration from about 0.1% w/v to about 0.2% w/v. In certain embodiments, brimonidine is used in combination with one or more active ingredients. In certain embodiments, brimonidine is present in the topical ophthalmic compositions of the invention as the sole active ingredient. The term “active ingredient” as used herein refers to a component of a composition which is responsible for the physiologic or therapeutic effect of composition in the amount present in the compositions of the present invention, whereas the other components of the composition (e.g., excipients, carriers, and diluents) are not responsible for a physiologic or therapeutic effect of composition in the amount present in the compositions of the present invention, even if they have other functions in the composition which are necessary or desired as part of the formulation (such as lubrication, pH control, emulsification, stabilization, preservation, and other functions other than the effect of composition as described herein). In some embodiments, compositions described herein in which brimonidine is the sole active ingredient which has therapeutic activity are compositions in which there are no other components which would be considered to have therapeutic activity for the treatment of ocular conditions (e.g., presbyopia) or improvement of vision parameters (e.g., near vision or near reading speed).

In certain embodiments, the brimonidine in the topical ophthalmic compositions of the present invention is present as a pharmaceutically acceptable salt or as a free base. As used herein, the term “pharmaceutically acceptable salts” refers to salts of brimonidine that are substantially non-toxic to living organisms, e.g., subjects in need of the topical ophthalmic compositions. Typical pharmaceutically acceptable salts include those salts prepared by reaction of the brimonidine with an inorganic or organic acid, or an organic base.

Inorganic acids which may be used to prepare pharmaceutically acceptable salts of brimonidine include, but are not limited to, hydrochloric acid, phosphoric acid, sulfuric acid, hydrobromic acid, hydroiodic acid, phosphorous acid and the like. Organic acids which may be used to prepare pharmaceutically acceptable salts of brimonidine include, without limitation, aliphatic mono- and dicarboxylic acids, such as tartaric acid, oxalic acid, carbonic acid, citric acid, succinic acid, phenyl-heteroatom-substituted alkanoic acids, aliphatic and aromatic sulfuric acids and the like. Pharmaceutically acceptable salts prepared from inorganic or organic acids thus include, but are not limited to, hydrochloride, hydrobromide, nitrate, sulfate, pyrosulfate, bisulfate, sulfite, bisulfite, phosphate, monohydrogenphosphate, dihydrogenphosphate, metaphosphate, pyrophosphate, hydroiodide, hydrofluoride, acetate, propionate, formate, oxalate, citrate, lactate, p-toluenesulfonate, methanesulfonate, and maleate. Suitable pharmaceutically acceptable salts may also be formed by reacting the active components with an organic base such as methylamine, ethylamine, ethanolamine, lysine, ornithine and the like. Pharmaceutically acceptable salts include the salts formed between carboxylate or sulfonate groups that may be found on some of the active components and inorganic cations, such as sodium, potassium, ammonium, or calcium, or such organic cations as isopropylammonium, trimethylammonium, tetramethylammonium, and imidazolium. All of these salts may be prepared by conventional means from the active components of the invention by reacting, for example, the appropriate acid or base with the active components of the invention. In specific aspects, the brimonidine is present in the topical ophthalmic compositions of the invention as brimonidine tartrate.

In certain embodiments, the brimonidine tartrate is present at a concentration from about 0.01% (w/v) to about 10% (w/v). In other embodiments, the brimonidine tartrate is present at a concentration from about 0.05% (w/v) to about 0.25% (w/v), about 0.075% (w/v) to about 0.175% (w/v), and 0.100% (w/v) to about 0.150% (w/v). In other embodiments, the brimonidine tartrate is present at a concentration from about 0.1% (w/v) to about 0.2% (w/v). In some embodiments, the brimonidine tartrate is present at a concentration of 0.15% (w/v). In specific embodiments, the brimonidine tartrate is present at a concentration of 0.1% (w/v). Other amounts of brimonidine tartrate that may be used include 0.01% (w/v), 0.02% (w/v), 0.025% (w/v) 0.03% (w/v), 0.04% (w/v), 0.05% (w/v), 0.06% (w/v), 0.07% (w/v), 0.08% (w/v), 0.09% (w/v), 0.095% (w/v), 0.099% (w/v), 0.10% (w/v), 0.11% (w/v), 0.12% (w/v), 0.125% (w/v), 0.13% (w/v), 0.135% (w/v), 0.14% (w/v), 0.145% (w/v), 0.15% (w/v), 0.155% (w/v), 0.16% (w/v), 0.17% (w/v), 0.18% (w/v), 0.19% (w/v), 0.195% (w/v), 0.20% (w/v), 0.205% (w/v), 0.21% (w/v), 0.22% (w/v), 0.23% (w/v), 0.24% (w/v), 0.25% (w/v), 0.26% (w/v), and ranges and amounts between any of these selected amounts of brimonidine tartrate.

The topical ophthalmic compositions of the invention further include a suitable buffer. As used herein, the term “buffer” refers to a component of a solution that resists changes in pH of the solution when an acid or alkali is added to it. Buffers typically involve a weak acid or alkali together with one of its salts. For example, a buffer may comprise one or more of sodium phosphate dibasic heptahydrate, sodium phosphate monobasic monohydrate, sodium hydroxide, or hydrochloric acid. In certain embodiments, the buffer comprises monobasic and dibasic sodium phosphate. The quality of a buffer is determined by its buffer capacity, i.e. its resistance to changes in pH when strong acids or bases are added. In other words, the buffer capacity corresponds to the amount of H⁺ or OH⁻ ions that can be neutralized by the buffer. Buffer capacity is related to the buffer concentration. A graph described by the relation of the pH to the addition of H⁺/OH⁻ ions is called the titration curve. The point of inflection of the curve corresponds to the pKa value of the buffer. The buffer capacity of a buffer is at its maximum at the pKa value. The pKa value of a buffer therefore corresponds to the mid-point of the pH range covered by the buffer and represents the point at which the concentration of acid and base is the same. In the area of this pH range, therefore, relatively large amounts of H⁺/OH⁻ ions result in only small changes in pH. Therefore, a buffer with more than one pKa resists changes to the pH of a solution over a broad range of H⁺/OH⁻ ions. Examples of buffers with more than one pKa include, but are not limited to, citrate buffer and phosphate buffer.

In certain embodiments, the buffer is present at a concentration of less than about 0.001% (w/v). In other embodiments, the buffer is present at a concentration of at least about 0.001% (w/v). In other embodiments, the buffer is present at a concentration from about 0.001% (w/v) to about 5% (w/v). In certain embodiments, the buffer is selected from the group consisting of phosphate, borate, borate citrate, sodium citrate dehydrate, and lactate buffer. In specific embodiments, the buffer is a borate buffer. In additional embodiments, the borate buffer is sodium borate decahydrate buffer. In certain aspects, the sodium borate decahydrate buffer is present at a concentration from about 0.01% (w/v) to about 0.1% (w/v). In specific aspects, the sodium borate decahydrate buffer is present at a concentration of about 0.045% (w/v).

A buffer may control the pH of the topical ophthalmic compositions of the invention. In certain embodiments, the topical ophthalmic compositions of the invention have a pH of about 1.0, about 1.5, about 2.0, about 2.5, about 3.0, about 3.5, about 4.0, about 4.5, about 5.0, about 5.5, about 6.0, about 6.5, about 7.0, about 7.5, or about 8.0. In other embodiments, the topical ophthalmic compositions of the invention have a pH of lower than about 8.0, lower than about 7.5, lower than about 7.0, lower than about 6.5, lower than about 6.0, lower than about 5.5, lower than about 5.0, lower than about 4.5, lower than about 4.0, lower than about 3.5, lower than about 3.0, lower than about 2.5, lower than about 2.0, or lower than about 1.5. In certain aspects, the topical ophthalmic compositions of the invention have a pH in the range of about 1.0 to about 8.0, about 1.5 to about 8.0, about 2.0 to about 8.0, about 2.5 to about 8.0, about 3.0 to about 8.0, about 3.5 to about 8.0, about 4.0 to about 8.0, about 4.5 to about 8.0, about 5.0 to about 8.0, about 5.5 to about 8.0, about 6.0 to about 8.0, about 6.5 to about 8.0, about 7.0 to about 8.0, or about 7.5 to about 8.0. In other aspects, the topical ophthalmic compositions of the invention have a pH in the range of about 3.0 to about 7.5, about 3.0 to about 7.0, about 3.0 to about 6.5, about 3.0 to about 6.0, about 3.0 to about 5.5, about 3.0 to about 5.0, about 3.0 to about 4.5, about 3.0 to about 4.0, or about 3.0 to about 3.5. In some aspects, the topical ophthalmic compositions of the invention have a pH of about 3.0 to about 8.0. In other aspects, the topical ophthalmic compositions of the invention have a pH of about 3.0 to about 5.5. In certain embodiments, the topical ophthalmic compositions of the invention have a pH of at least about 5.0. In other embodiments, the topical ophthalmic compositions of the invention have a pH of at least about 6.6. In yet other embodiments, the topical ophthalmic compositions of the invention have a pH of about 6.6 to about 8.0. In additional embodiments, the topical ophthalmic compositions of the invention have a pH of about 7.4 to about 8.0. In specific embodiments, the topical ophthalmic compositions of the invention have a pH of 7.7

The topical ophthalmic compositions of the invention may further comprise one or more viscosity enhancers. As used herein, the term “viscosity” of a topical ophthalmic composition of the invention is used as it normally is used for liquids and means a measure of the liquid's resistance to deformation at a given rate. Thus, viscosity is a quantity expressing the magnitude of internal friction, as measured by the force per unit area resisting a flow in which parallel layers of the topical ophthalmic compositions, unit distance apart, have unit speed relative to one another. A fluid that has no resistance to shear stress is known as an ideal or inviscid fluid. Zero viscosity is observed only at very low temperatures in superfluids. Otherwise, the second law of thermodynamics requires all fluids to have positive viscosity; such fluids are technically said to be viscous or viscid. A fluid with a relatively high viscosity, such as pitch, may appear to be a solid. In certain embodiments, the topical ophthalmic compositions of the invention have a viscosity from about 1 centipoise (cps) to about 10 cps. In certain aspects, the topical ophthalmic compositions of the invention have a viscosity close to that of pure water (1 cps). In specific aspects, the topical ophthalmic compositions of the invention have a viscosity of about 1 cps.

As used herein, the term “viscosity enhancer” refers to any substance that increases the viscosity of the topical ophthalmic compositions of the invention. A viscosity enhancer may be a polymer including, but not limited to hypromellose, carboxymethyl cellulose, hydroxyethyl cellulose, hydroxymethyl cellulose, methylcellulose, methyl cellulose 4000, hydroxypropyl cellulose, hydroxypropylmethyl cellulose, hydroxyl propyl methyl cellulose 2906, carboxypropylmethyl cellulose, hydroxypropylethyl cellulose, and hydroxyethyl cellulose, polyethylene glycol, polyvinyl alcohol, pyrrolidone, polyvinyl pyrrolidone, gellan, carrageenan, alignic acid, carboxyvinyl polymer, glycerol, acrylic polymers (e.g., carbomer, polycarbophil), or combinations thereof. In other aspects, a viscosity enhancer may be non-polymeric including, without limitation, hydroxypropyl-guar (hp-guar), xanthan gum, alginate, chitosan, gelrite, hyaluronic acid, dextran, or combinations thereof. In certain embodiments, the viscosity enhancer is present at a concentration of less than about 0.01% (w/v). In other embodiments, the viscosity enhancer is present at a concentration of at least about 0.01% (w/v). In certain aspects, the viscosity enhancer is present at a concentration from about 0.01% (w/v) to about 20% (w/v). In other aspects, the viscosity enhancer is present at a concentration from about 0.01% (w/v) to about 10% (w/v). In certain embodiments, the viscosity enhancer is carboxymethyl cellulose. In specific embodiments, the carboxymethyl cellulose is present at a concentration of about 0.5% (w/v).

The topical ophthalmic compositions of the invention may further include one or more osmolality agents in an amount that renders the topical ophthalmic compositions of the invention roughly isotonic. “Osmolality” is a measure of the total number of dissolved particles in a given volume of a solution. As used here, the term “osmolality agent” include any compound or substance useful for adjusting the osmolality of a topical ophthalmic composition. Any suitable ophthalmically acceptable osmolality agent may be used, provided that such component or components are compatible with the other ingredients of the topical ophthalmic compositions of the invention and do not have deleterious or toxic properties which could harm the subject to whom the compositions are administered. Examples of osmolality agents include, but are not limited to, salts, particularly sodium chloride or potassium chloride, organic compounds such as propylene glycol, mannitol, sorbitol, dextrose, and glycerin. In certain embodiments, the osmolality agents of the topical ophthalmic compositions of the invention include, but are not limited to, glycerin, propylene glycol, mannitol, sorbitol, sodium chloride, potassium chloride and dextrose.

“Tonicity” is a measure of the effective osmotic pressure gradient, as defined by the water potential of two solutions separated by a semipermeable membrane. In other words, tonicity is the relative concentration of solutes dissolved in solution which determine the direction and extent of diffusion. The term is commonly used when describing the response of cells immersed in an external solution. Unlike osmotic pressure, tonicity is influenced only by solutes that cannot cross the membrane, as only these exert an effective osmotic pressure. Solutes able to freely cross the membrane do not affect tonicity because they will always be in equal concentrations on both sides of the membrane. There are three classifications of tonicity that one solution can have relative to another: hypertonic, hypotonic, and isotonic. A solution is “isotonic” when its effective osmole concentration is the same as that of another solution. In biology, the solutions on either side of a cell membrane, for example, are isotonic if the concentration of solutes outside the cell is equal to the concentration of solutes inside the cell.

In certain embodiments, the one or more osmolality agents is selected from the group consisting of glycerin, propylene glycol, mannitol, sorbitol, sodium chloride, potassium chloride, and dextrose. The amount of an osmolality agent may vary depending upon whether the topical ophthalmic compositions are isotonic, hypertonic, or hypotonic. In certain embodiments, the amount of an osmolality agent such as those listed above may be at least about 0.0001% (w/v) up to about 1% (w/v), about 2% (w/v), about 5% (w/v), about 10% (w/v), or about 20% (w/v). In some embodiments, at least one of the one or more osmolality agents is present at a concentration of at least about 0.0001% (w/v). In other embodiments, the one or more osmolality agents are each present at a concentration of at least about 0.0001% (w/v). In some embodiments, at least one of the one or more osmolality agents is present at a concentration from about 0.001% (w/v) to about 20% (w/v). In other embodiments, the one or more osmolality agents are each present at a concentration from about 0.001% (w/v) to about 20% (w/v). In additional embodiments, at least one of the one or more osmolality agents is present at a concentration from about 0.001% (w/v) to about 5% (w/v). In further embodiments, the one or more osmolality agents are each present at a concentration from about 0.001% (w/v) to about 5% (w/v). In yet other embodiments, at least one of the one or more osmolality agents is present at a concentration from about 0.001% (w/v) to about 2.5% (w/v). In additional embodiments, the one or more osmolality agents are each present at a concentration from about 0.001% (w/v) to about 2.5% (w/v). In still other embodiments, at least one of the one or more osmolality agents is present at a concentration from about 0.001% (w/v) to about 1% (w/v). In further embodiments, the one or more osmolality agents are each present at a concentration from about 0.001% (w/v) to about 1% (w/v). In certain embodiments, the topical ophthalmic compositions of the invention further comprise calcium chloride and magnesium chloride.

The topical ophthalmic compositions of the invention may further include a strong acid or a strong base. Examples of strong acids and strong bases are well known in the art and include, without limitation, NaOH, KOH, HCl, and H₂SO₄. In specific aspects, the topical ophthalmic compositions of the invention further comprise NaOH or HCl

The topical ophthalmic compositions of the invention may also include boric acid. In certain embodiments, the boric acid is present at a concentration from about 0.01% (w/v) to about 5% (w/v). In additional embodiments, the boric acid is present at a concentration from about 0.1% (w/v) to about 1.5% (w/v). In certain embodiments, the boric acid is present at a concentration of about 1% (w/v)

The topical ophthalmic compositions of the invention may be packaged for single use, and contain no preservative or essentially no preservative. Alternatively, the topical ophthalmic compositions of the invention may be packaged for multiple uses, and comprise a suitable preservative to prevent contamination over multiple uses. As used herein, the term “preservative” means any substance that prevents or retards contamination in the form of bacterial or fungal growth in the topical ophthalmic solutions of the invention. Examples of suitable preservatives include, but are limited to, benzalkonium chloride (BAK), Polyquaternium-1 (Polyquad®), chlorobutanol, stabilized chlorine dioxide, and a stabilized oxychloro complex comprising chlorite, chlorate and chlorine dioxide. Stabilized oxychloro complex or stabilized chlorine dioxide, also known as Purite®, may be described as an aqueous solution of sodium chlorite (NaClO₂). U.S. Pat. No. 5,424,078, which is incorporated herein by reference in its entirety, further discusses the use of stabilized chlorine dioxide as a preservative for topical ophthalmic compositions.

In certain embodiments, the preservative is present at a concentration of at least about 1 ppm. In other embodiments, the preservative is present at a concentration of less than about 1 ppm. In some aspects, the preservative is present at a concentration from about 1 ppm to about 1000 ppm. In other aspects, the preservative is present at a concentration from about 10 ppm to about 300 ppm. In other embodiments, the preservative is present at a concentration from about 10 ppm to about 200 ppm. In certain aspects, the preservative is present at a concentration of less than about 0.001% (w/v). In other aspects, the preservative is present at a concentration of at least about 0.001% (w/v). In certain embodiments, the preservative is present at a concentration from about 0.001% (w/v) to about 1% (w/v). In certain aspects, the preservative is a stabilized oxychloro complex. In some aspects, the stabilized oxychloro complex is present at a concentration from about 0.001% (w/v) to about 0.02% (w/v). In specific aspects, the stabilized oxychloro complex is present at a concentration of about 0.005% (w/v).

The topical ophthalmic compositions of the invention may or may not contain a secondary buffering agent. In certain aspects, the secondary buffering agent includes, without limitation, citrate buffer and acetate buffer. In certain embodiments, the secondary buffering agent is present at a concentration of at least about concentration of less than about 0.001 mM. In other embodiments, the secondary buffering agent is present at a concentration of at least about 0.001 mM. In some embodiments, the secondary buffering agent is present at a concentration from about 0.01 mM to 1 M. In specific embodiments, the secondary buffering agent is present at a concentration from about 1 mM to about 100 mM.

In certain embodiments, the topical ophthalmic compositions of the invention comprise 0.1% (w/v) brimonidine, 0.5% (w/v) carboxymethyl cellulose, 0.005% (w/v) stabilized oxychloro complex, 0.6% (w/v) boric acid, 0.045% (w/v) sodium borate decahydrate, 0.37% (w/v) sodium chloride, 0.14% (w/v) potassium chloride, 0.006% (w/v) calcium chloride, 0.006% (w/v) magnesium chloride, and NaOH and/or HCl, wherein the topical ophthalmic compositions have a pH of 7.7.

The topical ophthalmic compositions of the invention may be prepared by techniques known to those skilled in the art. The topical ophthalmic compositions of the invention may be an aqueous solution, emulsion or suspension or may be a dried preparation. In some aspects, the topical ophthalmic compositions of the invention may be desiccated or lyophilized, for example, by freeze-drying or spray drying for storage or formulations purposes. In certain aspects, a solid composition of the invention is subsequently reconstituted into liquid compositions by the addition of an appropriate liquid carrier prior to administering to a subject in need thereof.

The invention further relates to methods of treating an ocular condition in a subject in need of treatment thereof, comprising administering a topical ophthalmic composition comprising brimonidine. As used herein, the term “ocular condition” may refer to any condition, disease, or impairment, which affects or involves the eye or one of the parts or regions of the eye, and includes optical issues causing refractive errors in the eye. Ocular conditions include, but are not limited to presbyopia, myopia, progressive myopia, pathologic myopia, amblyopia, cycloplegia, mydriasis, allergic conjunctivitis, conjunctival hyperemia, red eye, glaucoma, ocular hypertension, night vision symptoms post refractive surgery (e.g., glare, halos or starbursts around lights), accommodative esotropia, glaucoma, ocular hypertension, accommodative insufficiency, hyperopia, anisocoria, astigmatism, amblyopia, Adie's tonic pupil, or other causes of parasympathetic denervation, complications arising after refractive surgery, such as decentered ablations following LASIK or PRK, LASIK undercorrections, LASIK overcorrections, corneal scars, hazing, and refractive errors. In specific embodiments, the ocular condition is presbyopia.

The invention further provides methods of treating presbyopia in a subject in need of treatment thereof. The methods comprise administering to at least one eye of the subject a therapeutically effective amount of a topical ophthalmic composition comprising brimonidine.

“Presbyopia” is farsightedness often caused by loss of elasticity of the lens of the eye, occurring typically in middle and old age. Presbyopia is a condition associated with the aging of the eye that results in progressively worsening ability to focus clearly (particularly at close distance). Symptoms include difficulty reading small print, having to hold reading material farther away, headaches, and eyestrain. Most people begin to notice the effects of presbyopia sometime after age 40, when they start having trouble seeing small print clearly—including text messages on their phone. Application of cholinergic agonists (miotic agents) in these subjects is beneficial as the miosis resulting from sphincter muscle contraction creates a “pin-hole effect” that may potentially improve the near and intermediate vision by increasing the depth of field. These cholinergic agonists can thus be used for the treatment of presbyopia, although most effective dosing frequency and dose concentrations have not been defined.

As used herein, the term “treating” refers to both therapeutic measures and prophylactic or preventative measures, wherein the objective is to prevent, slow down (lessen), or ameliorate the progression of a disease (e.g., presbyopia). Beneficial or desired clinical results include, but are not limited to, alleviation of symptoms, diminishing the extent of the disease, stabilized (i.e., not worsening) state of the disease, delaying or slowing of disease progression, amelioration or palliation of the disease state, and reversing the disease (whether partial or total).

As used herein, the term “subject” refers to any individual, e.g., a mammal, for whom diagnosis, prognosis, or therapy is desired. The term “subject” may mean a human or non-human mammal affected, likely to be affected, or suspected to be affected with an ocular condition or disease. Although the topical ophthalmic compositions provided herein are principally directed to compositions which are suitable for administration to humans, the skilled artisan will understand that such compositions are generally suitable for administration to subjects of all sorts. In certain aspects, the subject is a mammal. In some aspects, a mammal includes, without limitation, primates, such as humans, monkeys and apes, and non-primates such as domestic animals, including laboratory animals, sports animals, farm animals, and household pets (e.g., cats, dogs, swine, cattle, cows, sheep, goats, horses, guinea pigs, rabbits, rats, mice), and non-domestic animals, such as wildlife, birds, or the like. In certain embodiments, the subject was not previously diagnosed with glaucoma.

As used herein, the term “a subject in need thereof” includes subjects, such as mammalian subjects, that would benefit from administration of a topical ophthalmic composition of the invention. Subjects in need of treatment include, without limitation, those already with the condition or disorder as well as those prone to having the condition or disorder, or those in which the condition or disorder is to be prevented, ameliorated, or reversed.

By “therapeutically effective” is meant that the topical ophthalmic compositions are able to exert a statistically significant medically beneficial effect when used as prescribed or directed, as compared to a placebo.

The term “administer,” or “administering” as it applies to, for example, a subject in need of the topical ophthalmic compositions of the invention, refers to contact of, for example, the topical ophthalmic composition of the invention to at least one eye of the subject. In the context of a cell, administration includes contact (e.g., in vitro or ex vivo) of the topical ophthalmic compositions of the invention to the cell, as well as contact of the topical ophthalmic compositions of the invention with a fluid, where the fluid is in contact with the cell. In certain embodiments, the topical ophthalmic compositions of the invention are administered to only one eye of a subject in need thereof. In other embodiments, the topical ophthalmic compositions of the invention are administered to at least one eye of a subject. In yet other embodiments, the topical ophthalmic compositions of the invention are administered to both eyes of a subject.

Normally a subject has a dominant eye and a non-dominant eye. The “dominant eye” is the eye that has a greater visual acuity and, therefore, dominates the depth vision. The “non-dominant eye” usually dominates the peripheral and spatial vision. Their interaction causes the brain to receive a three-dimensional image. Usually the dominant eye is the eye that is used to look through a microscope, a camera, or for any task in which only one eye is used. In certain embodiments, the topical ophthalmic compositions of the invention are administered to a non-dominant eye of a subject. In other embodiments, the topical ophthalmic compositions of the invention are administered to a dominant eye of a subject. In yet other embodiments, the topical ophthalmic compositions of the invention are administered to both the non-dominant eye and the dominant eye of the subject.

The topical ophthalmic compositions of the invention may be administered at several intervals in order to sustain therapeutic levels. For example, the topical ophthalmic compositions of the invention may be administered once daily, twice daily (BID), three times daily (TID), four times daily (QID) or more. In some embodiments, the topical ophthalmic compositions of the invention are administered at least once daily. In specific embodiments, the topical ophthalmic compositions of the invention are administered three times daily.

In certain aspects, the topical ophthalmic compositions of the invention have a duration of action of at least about 1 hour, at least about 2 hours, at least about 3 hours, at least about 4 hours, at least about 5 hours, at least about 6 hours, at least about 7 hours, at least about 8 hours, at least about 9 hours, at least about 10 hours, at least about 11 hours, at least about 12 hours, at least about 24 hours, as well as all intervening time points. In specific embodiments, the topical ophthalmic compositions of the invention have a duration of action greater than 10 hours, for example, 12 hours, or even 24 hours. As used herein the term “duration of action” refers to the duration of time that an administered topical ophthalmic composition has an effect on at least one vision parameter (e.g., improvement of near vision or improvement of near reading speed), or ocular condition (e.g., presbyopia), or reduction of pupil diameter in a subject in need thereof. In certain embodiments, the topical ophthalmic compositions of the invention remain effective following administration for a period of time selected from the group consisting of at least about 6 hours, at least about 8 hours, at least about 10 hours, at least about 12 hours, and at least about 24 hours.

The invention additionally provides a method of treating presbyopia in a subject in need of treatment thereof, comprising administering to at least one eye of the subject a therapeutically effective amount of a topical ophthalmic composition comprising 0.1% (w/v) brimonidine, 0.5% (w/v) carboxymethyl cellulose, 0.005% (w/v) stabilized oxychloro complex, 0.6% (w/v) boric acid, 0.045% (w/v) sodium borate decahydrate, 0.37% (w/v) sodium chloride, 0.14% (w/v) potassium chloride, 0.006% (w/v) calcium chloride, 0.006% (w/v) magnesium chloride, and NaOH and/or HCl, wherein the topical ophthalmic composition has a pH of 7.7.

The invention further provides methods for improvement of near vision in a subject with presbyopia in need of treatment thereof. The methods comprise administering to at least one eye of the subject a therapeutically effective amount of a topical ophthalmic composition comprising brimonidine. In some embodiments, methods for improving vision (e.g., near vision) and/or treating a disease (e.g., presbyopia) may be administered to a subject in need thereof with an age greater than 40 years old, between 40 and 50 years old, greater than 50 years old, greater than 60 years old, and ranges and specific ages therebetween. In a preferred embodiment, methods and compositions disclosed herein are administered to a subject aged greater than 50 years old.

In certain embodiments, the methods of the invention result in an increase of mean near log MAR thresholds of at least about 0.1 from baseline within about 5 minutes, about 6 minutes, about 7 minutes, about 8 minutes, about 9 minutes, about 10 minutes, about 11 minutes, about 12 minutes, about 13 minutes, about 14 minutes, about 15 minutes, about 30 minutes, about 45 minutes, about 60 minutes, about 90 minutes, or about 120 minutes following administration of the topical ophthalmic compositions of the invention. In some embodiments, the methods of the invention result in an increase of mean near log MAR thresholds of at least about 0.1 from baseline within about 5 minutes to about 15 minutes following administration of the topical ophthalmic compositions of the invention. In certain aspects, the methods of the invention result in an increase of mean near log MAR thresholds of at least about 0.1 from baseline within about 15 minutes following administration of the topical ophthalmic compositions of the invention. In specific aspects, the methods of the invention result in an increase of mean near log MAR thresholds of at least about 0.1 from baseline within about 5 minutes following administration of the topical ophthalmic compositions of the invention. In additional embodiments, the methods of the invention result in an increase of mean near log MAR thresholds of about 0.5 from baseline within about 5 minutes, about 6 minutes, about 7 minutes, about 8 minutes, about 9 minutes, about 10 minutes, about 11 minutes, about 12 minutes, about 13 minutes, about 14 minutes, about 15 minutes, about 30 minutes, about 45 minutes, about 60 minutes, about 90 minutes, or about 120 minutes following administration of the topical ophthalmic compositions of the invention. In some embodiments, the methods of the invention result in an increase of mean near log MAR thresholds of about 0.5 from baseline within about 5 minutes to about 15 minutes following administration of the topical ophthalmic compositions of the invention. In certain aspects, the methods of the invention result in an increase of mean near log MAR thresholds of about 0.5 from baseline within about 15 minutes following administration of the topical ophthalmic compositions of the invention. In specific aspects, the methods of the invention result in an increase of mean near log MAR thresholds of about 0.5 from baseline within about 5 minutes following administration of the topical ophthalmic compositions of the invention.

In some embodiments, the methods of the invention result in an improvement in acuity of at least 0.1 log MAR in at least about 25% subjects within about 15 minutes following administration of the topical ophthalmic compositions of the invention under mesopic conditions (e.g., at 20 lux). In certain aspects, the methods of the invention result in an improvement in acuity of at least 0.1 log MAR in about 50% subjects within about 15 minutes following administration of the topical ophthalmic compositions of the invention under mesopic conditions (e.g., at 20 lux). As used herein, the term “mesopic” vision refers to a combination of photopic vision and scotopic vision in low but not quite dark lighting situations. Mesopic light levels range from luminances of approximately 0.001 to 3 cd m⁻². Most night-time outdoor and traffic lighting scenarios are in the mesopic range. The human eye uses scotopic vision under low-light conditions and mesopic vision in intermediate conditions. Humans see differently at different light levels. This is because under high light levels typical during the day (photopic vision), the eye uses cones to process light. Under very low light levels, corresponding to moonless nights without electric lighting (scotopic vision), the eye uses rods to process light. At many night-time levels, a combination of both cones and rods supports vision. Photopic vision facilitates excellent color discrimination ability, whereas colors are indiscriminable under scotopic vision. Mesopic vision falls between these two extremes. In most night-time environments, there is enough ambient light at night to prevent true scotopic vision.

In additional embodiments, the methods of the invention result in an improvement in acuity of at least 0.1 log MAR in at least about 20% subjects within about 15 minutes following administration of the topical ophthalmic compositions of the invention under photopic conditions (e.g., at 200 lux). In certain aspects, the methods of the invention result in an improvement in acuity of at least 0.1 log MAR in about 50% subjects within about 15 minutes following administration of the topical ophthalmic compositions of the invention under photopic conditions (e.g., at 200 lux). In other embodiments, the methods of the invention result in an improvement in acuity of at least 0.1 log MAR in at least about 5% subjects within about 15 minutes following administration of the topical ophthalmic compositions of the invention under highly photopic conditions (e.g., at 2000 lux). In certain aspects, the methods of the invention result in an improvement in acuity of at least 0.1 log MAR in about 10% subjects within about 15 minutes following administration of the topical ophthalmic compositions of the invention under highly photopic conditions (e.g., at 2000 lux). In yet other embodiments, the methods of the invention result in an improvement in acuity of at least 0.2 log MAR in about 30% subjects within about 15 minutes following administration of the topical ophthalmic compositions of the invention under mesopic conditions (e.g., at 20 lux). In still other embodiments, the methods of the invention result in an improvement in acuity of at least 0.2 log MAR in about 20% subjects within about 15 minutes following administration of the topical ophthalmic compositions of the invention under photopic conditions (e.g., at 200 lux).

As used herein, the term “photopic” vision is the vision of the eye under well-lit conditions (luminance level 10 to 10⁸ cd/m²). In humans and other animals, photopic vision allows color perception, mediated by cone cells, and a significantly higher visual acuity and temporal resolution than available with scotopic vision (the vision of the eye under low-light conditions; luminance level 10⁻³ to 10⁻⁶ cd/m²).

The invention additionally provides methods of improving near reading speed in a subject with presbyopia in need of treatment thereof. The methods comprise administering to at least one eye of the subject a therapeutically effective amount of a topical ophthalmic composition comprising brimonidine.

In certain embodiments, the methods of the invention allow subjects suffering from presbyopia to proficiently read typical newspapers and cellphone messages at room light levels. In certain aspects, for reading typical newspapers and cellphone messages, the absolute reading speed achieved with the topical ophthalmic compositions of the invention is greater than about 100 words per minute at room light levels (e.g., 125 words per minute at 200 lux). In other embodiments, the methods of the invention allow subjects suffering from presbyopia to proficiently read typical newspapers and cellphone message outdoors. In certain aspects, for reading typical newspapers and cellphone messages, the absolute reading speed achieved with the topical ophthalmic compositions of the invention is greater than about 100 words per minute outdoors (e.g., 140 words per minute at 2000 lux). In additional embodiments, the methods of the invention allow subjects suffering from presbyopia to proficiently read text seen in grocery labels outdoors. In certain aspects, for reading text seen in grocery labels, the absolute reading speed achieved with the topical ophthalmic compositions of the invention is greater than about 100 words per minute outdoors (e.g., 128 words per minute at 2000 lux). In further embodiments, the methods of the invention allow subjects suffering from presbyopia to proficiently read text seen in over-the-counter (OTC) labels outdoors. In certain aspects, for reading text seen in OTC labels, the absolute reading speed achieved with the topical ophthalmic compositions of the invention is greater than about 100 words per minute outdoors (e.g., 103 words per minute at 2000 lux).

In certain embodiments, the methods of the invention result in an absolute reading speed of at least about 64 words per minute, at least about 125 words per minute, and at least about 140 words per minute for text size commonly seen in US newspaper and cellphone text message within about 15 minutes following administration of the topical ophthalmic compositions of the invention at light levels of 20, 200, and 2000 lux, respectively. In other embodiments, the methods of the invention result in an absolute reading speed of at least about 40 words per minute, at least about 94 words per minute, and at least about 128 words per minute for text size of grocery labels within about 15 minutes following administration of the topical ophthalmic compositions of the invention at light levels of 20, 200, and 2000 lux, respectively. In additional embodiments, the methods of the invention result in an absolute reading speed of at least about 21 words per minute, at least about 52 words per minute, and at least about 103 words per minute for text size of over-the-counter (OTC) drugs within about 15 minutes following administration of the topical ophthalmic compositions of the invention at light levels of 20, 200, and 2000 lux, respectively.

In specific embodiments, the methods of the invention result in an improvement of average reading speed across a font size range from about 0.25 log MAR to about 0.6 log MAR by at least about 17 words per minute from baseline under mesopic conditions within about 15 minutes following administration of the topical ophthalmic composition.

In additional embodiments, the methods of the invention result in an improvement of average reading speed across a font size range from about 0.25 log MAR to about 0.6 log MAR by at least about 10 words per minute from baseline under photopic conditions within about 5 minutes, about 6 minutes, about 7 minutes, about 8 minutes, about 9 minutes, about 10 minutes, about 11 minutes, about 12 minutes, about 13 minutes, about 14 minutes, about 15 minutes, about 30 minutes, about 45 minutes, about 60 minutes, about 90 minutes, or about 120 minutes following administration of the topical ophthalmic composition. In some embodiments, the methods of the invention result in an improvement of average reading speed across a font size range from about 0.25 log MAR to about 0.6 log MAR by at least about 10 words per minute from baseline under photopic conditions within about 5 minutes to about 15 minutes following administration of the topical ophthalmic composition. In certain aspects, the methods of the invention result in an improvement of average reading speed across a font size range from about 0.25 log MAR to about 0.6 log MAR by at least about 10 words per minute from baseline under photopic conditions within about 15 minutes following administration of the topical ophthalmic compositions of the invention. In specific aspects, the methods of the invention result in an improvement of average reading speed across a font size range from about 0.25 log MAR to about 0.6 log MAR by at least about 10 words per minute from baseline under photopic conditions within about 5 minutes following administration of the topical ophthalmic compositions of the invention.

The invention also provides methods of reducing pupil diameter in a subject with presbyopia in need of treatment thereof. The methods comprise administering to at least one eye of the subject a therapeutically effective amount of a topical ophthalmic composition comprising brimonidine.

The normal pupil size in adults varies from 2 to 4 mm in diameter in bright light to 4 to 8 mm in the dark. The pupils are generally equal in size. They constrict to direct illumination (direct response) and to illumination of the opposite eye (consensual response). The pupil dilates in the dark. Both pupils constrict when the eye is focused on a near object (accommodative response).

In certain embodiments, the methods of the invention result in a reduction of pupil diameter of at least about 10% of baseline pupil diameter over a time period of about 10 minutes to about 180 minutes following administration of the topical ophthalmic compositions of the invention. In other embodiments, the methods of the invention result in a reduction of pupil diameter of at least about 80% of baseline pupil diameter over a time period of about 10 minutes to about 180 minutes following administration of the topical ophthalmic compositions of the invention. In additional embodiments, the methods of the invention result in a reduction of pupil diameter of about 10% to about 90% of baseline pupil diameter over a time period of about 10 minutes to about 180 minutes following administration of the topical ophthalmic compositions of the invention. In specific embodiments, the methods of the invention result in a reduction of pupil diameter of about 20% to about 30% of baseline pupil diameter over a time period of about 30 minutes to about 120 minutes following administration of the topical ophthalmic compositions of the invention. In other embodiments, the methods of the invention result in a reduction of pupil diameter of about 10% of baseline pupil diameter at about 180 minutes following administration of the topical ophthalmic compositions of the invention.

In additional embodiments, the methods of the invention result in a reduction of pupil diameter from about 63% to about 68% of baseline within about 5 minutes, about 6 minutes, about 7 minutes, about 8 minutes, about 9 minutes, about 10 minutes, about 11 minutes, about 12 minutes, about 13 minutes, about 14 minutes, about 15 minutes, about 30 minutes, about 45 minutes, about 60 minutes, about 90 minutes, or about 120 minutes following administration of the topical ophthalmic compositions of the invention under highly mesopic conditions (e.g., at about 0 lux). In some aspects, the methods of the invention result in a reduction of pupil diameter from about 63% to about 68% of baseline within about 60 minutes following administration of the topical ophthalmic compositions of the invention under highly mesopic conditions. In other aspects, the methods of the invention result in a reduction of pupil diameter from about 63% to about 68% of baseline within about 5 minutes to about 15 minutes following administration of the topical ophthalmic compositions of the invention under highly mesopic conditions. In further embodiments, the methods of the invention result in a reduction of pupil diameter from about 66% to about 68% of baseline within about 5 minutes, about 6 minutes, about 7 minutes, about 8 minutes, about 9 minutes, about 10 minutes, about 11 minutes, about 12 minutes, about 13 minutes, about 14 minutes, about 15 minutes, about 30 minutes, about 45 minutes, about 60 minutes, about 90 minutes, or about 120 minutes following administration of the topical ophthalmic compositions of the invention under mesopic conditions (e.g., at about 20 lux). In some aspects, the methods of the invention result in a reduction of pupil diameter from about 66% to about 68% of baseline within about 60 minutes following administration of the topical ophthalmic compositions of the invention under mesopic conditions. In other aspects, the methods of the invention result in a reduction of pupil diameter from about 66% to about 68% of baseline within about 5 minutes to about 15 minutes following administration of the topical ophthalmic compositions of the invention under mesopic conditions. In other embodiments, the methods of the invention result in a reduction of pupil diameter of about 77% of baseline within about 5 minutes, about 6 minutes, about 7 minutes, about 8 minutes, about 9 minutes, about 10 minutes, about 11 minutes, about 12 minutes, about 13 minutes, about 14 minutes, about 15 minutes, about 30 minutes, about 45 minutes, about 60 minutes, about 90 minutes, or about 120 minutes following administration of the topical ophthalmic compositions of the invention under photopic conditions (e.g., at about 200 lux). In some aspects, the methods of the invention result in a reduction of pupil diameter of about 77% of baseline within about 60 minutes following administration of the topical ophthalmic compositions of the invention under photopic conditions. In some aspects, the methods of the invention result in a reduction of pupil diameter of about 77% of baseline within about 5 minutes to about 15 minutes following administration of the topical ophthalmic compositions of the invention under photopic conditions. In yet other embodiments, the methods of the invention result in a reduction of pupil diameter from about 77% to about 87% of baseline within about 5 minutes, about 6 minutes, about 7 minutes, about 8 minutes, about 9 minutes, about 10 minutes, about 11 minutes, about 12 minutes, about 13 minutes, about 14 minutes, about 15 minutes, about 30 minutes, about 45 minutes, about 60 minutes, about 90 minutes, or about 120 minutes following administration of the topical ophthalmic compositions of the invention under highly photopic conditions (e.g., at about 2000 lux). In some aspects, the methods of the invention result in a reduction of pupil diameter from about 77% to about 87% of baseline within about 60 minutes following administration of the topical ophthalmic compositions of the invention under highly photopic conditions. In other aspects, the methods of the invention result in a reduction of pupil diameter from about 77% to about 87% of baseline within about 5 minutes to about 15 minutes following administration of the topical ophthalmic compositions of the invention under highly photopic conditions.

In certain embodiments, the methods of the invention result in a reduction of pupil diameter of at least about 60% of baseline pupil diameter within about 5 minutes, about 6 minutes, about 7 minutes, about 8 minutes, about 9 minutes, about 10 minutes, about 11 minutes, about 12 minutes, about 13 minutes, about 14 minutes, about 15 minutes, about 30 minutes, about 45 minutes, about 60 minutes, about 90 minutes, or about 120 minutes following administration of the topical ophthalmic compositions of the invention. In some aspects, the methods of the invention result in a reduction of pupil diameter of at least about 60% of baseline pupil diameter within about 60 minutes following administration of the topical ophthalmic compositions of the invention. In other aspects, the methods of the invention result in a reduction of pupil diameter of at least about 60% of baseline pupil diameter within about 5 minutes to about 15 minutes following administration of the topical ophthalmic compositions of the invention. In other embodiments, the methods of the invention result in a reduction of pupil diameter of at least about 70% of baseline pupil diameter within about 5 minutes, about 6 minutes, about 7 minutes, about 8 minutes, about 9 minutes, about 10 minutes, about 11 minutes, about 12 minutes, about 13 minutes, about 14 minutes, about 15 minutes, about 30 minutes, about 45 minutes, about 60 minutes, about 90 minutes, or about 120 minutes following administration of the topical ophthalmic compositions of the invention. In some embodiments, the methods of the invention result in a reduction of pupil diameter of at least about 70% of baseline pupil diameter within about 5 minutes to about 15 minutes following administration of the topical ophthalmic compositions of the invention. In certain aspects, the methods of the invention result in a reduction of pupil diameter of at least about 70% of baseline pupil diameter within about 15 minutes following administration of the topical ophthalmic compositions of the invention. In specific aspects, the methods of the invention result in a reduction of pupil diameter of at least about 70% of baseline pupil diameter within about 5 minutes following administration of the topical ophthalmic compositions of the invention. In additional embodiments, the methods of the invention result in a reduction of pupil diameter of at least about 63% of baseline pupil diameter within about 5 minutes, about 6 minutes, about 7 minutes, about 8 minutes, about 9 minutes, about 10 minutes, about 11 minutes, about 12 minutes, about 13 minutes, about 14 minutes, about 15 minutes, about 30 minutes about 45 minutes, about 60 minutes, about 90 minutes, or about 120 minutes following administration of the topical ophthalmic compositions of the invention. In some embodiments, the methods of the invention result in a reduction of pupil diameter of at least about 63% of baseline pupil diameter within about 5 minutes to about 15 minutes following administration of the topical ophthalmic compositions of the invention. In some aspects, the methods of the invention result in a reduction of pupil diameter of at least about 63% of baseline pupil diameter within about 15 minutes following administration of the topical ophthalmic compositions of the invention. In specific aspects, the methods of the invention result in a reduction of pupil diameter of at least about 63% of baseline pupil diameter within about 5 minutes following administration of the topical ophthalmic compositions of the invention. In further embodiments, the methods of the invention result in a reduction of pupil diameter of about 80% of baseline pupil diameter within about 5 minutes, about 6 minutes, about 7 minutes, about 8 minutes, about 9 minutes, about 10 minutes, about 11 minutes, about 12 minutes, about 13 minutes, about 14 minutes, about 15 minutes, about 30 minutes about 45 minutes, about 60 minutes, about 90 minutes, or about 120 minutes following administration of the topical ophthalmic compositions of the invention. In some embodiments, the methods of the invention result in a reduction of pupil diameter of about 80% of baseline pupil diameter within about 60 minutes following administration of the topical ophthalmic compositions of the invention. In other embodiments, the methods of the invention result in a reduction of pupil diameter of about 80% of baseline pupil diameter within about 5 minutes to about 15 minutes following administration of the topical ophthalmic compositions of the invention. In some aspects, the methods of the invention result in a reduction of pupil diameter of about 80% of baseline pupil diameter within about 15 minutes following administration of the topical ophthalmic compositions of the invention. In specific aspects, the methods of the invention result in a reduction of pupil diameter of about 80% of baseline pupil diameter within about 5 minutes following administration of the topical ophthalmic compositions of the invention.

In certain embodiments, treatment with the topical ophthalmic composition retains distance visual acuity under mesopic conditions compared to baseline. In other embodiments, treatment with the topical ophthalmic composition retains distance visual acuity under photopic conditions compared to baseline. In additional embodiments, administering the topical ophthalmic compositions of the invention does not significantly reduce intraocular pressure in the eye of the subject.

The invention further provides methods of improving at least one vision parameter in a subject in need thereof, comprising administering to at least one eye of the subject one or more topical ophthalmic compositions of the invention. As used herein, the term “vision parameter” refers to any characteristic in a subject's vision that may be measured and is susceptible to being improved by the topical ophthalmic compositions and methods described herein. Vision parameters include, but are not limited to, near vision acuity, intermediate visual acuity, distance visual acuity, night vision, day vision, optical aberrations (e.g., glare, light scattering), and uncorrected refractive errors. Additional examples of vision parameters include, without limitation, night time glare, post-LASIK “star burst” glare, visual “halos” seen around light sources, and accommodative insufficiency.

“Improving vision parameter,” including, but not limited to, near, intermediate, and/or distance visual acuity, may for example be reflected in the increase of number of letters correctly read at any time point post dosing, the increase in the average letter change, or 2-line or 3-line improvement, all from baseline (i.e., from pre-treatment). Night vision improvement may be reflected in visual improvement for subjects in dim or dark lighting (e.g., under mesopic or scotopic conditions). Day vision improvement may be reflected in visual improvement for subjects in bright lighting as found during daylight hours or in sunshine (e.g., under photopic conditions). Vision improvement using the methods described herein may also be achieved in combination with or when using other visual aids and devices (e.g., those used for treating presbyopia), including but not limited to reading glasses, lens modifying medications, and surgical presbyopic options including intraocular lenses (IOLs).

In certain embodiments, methods of treatment using the topical ophthalmic compositions described herein result in an at least 2-line improvement from baseline under the condition of photopic, high contrast uncorrected near visual acuity (UNVA).

As used herein, the term “uncorrected near visual acuity” (UNVA) refers to a subject's ability, without any vision aid (such as eyeglasses or contact lenses), to see the details of objects within arm's distance from the body (e.g., at 33-41 cm away from the eye).

In some embodiments, methods of treatment using the topical ophthalmic compositions described herein result in an at least 3-line improvement from baseline under the condition of photopic, high contrast UNVA. In other embodiments, methods described herein result in an increase in the average letter change from baseline under the condition of photopic, high contrast UNVA.

The term “improvement from baseline” refers to the increase from pre-treatment in the number of letters correctly read at certain post treatment time point. As used herein, the term “2-line improvement from baseline” or “3-line improvement from baseline” or similar improvement from baseline refers to a subject's ability to read 2 or 3 more lines of letters on a standard chart (e.g., Snellen, ETDRS, Logarithmic Visual Acuity Chart, etc.) after treatment with a topical ophthalmic composition of the invention when comparing to the number of lines readable before treatment.

In certain embodiments, methods of treatment using the topical ophthalmic compositions described herein result in an at least 2-line improvement from baseline under the condition of mesopic, high contrast UNVA.

In some embodiments, methods of treatment using the topical ophthalmic compositions described herein result in an at least 3-line improvement from baseline under the condition of mesopic, high contrast UNVA. In other embodiments, methods described herein result in an increase in the average letter change from baseline under the condition of mesopic, high contrast UNVA.

In certain embodiments, methods of treatment using the topical ophthalmic compositions described herein result in an at least 2-line improvement from baseline under the condition of photopic, high contrast uncorrected distance visual acuity (UDVA). As used herein, the term “uncorrected distance visual acuity” (UDVA) refers to a subject's ability, without any vision aid (such as eyeglasses or contact lenses), to see the details of objects beyond arm's distance from the body (e.g., greater than 4 meters away from the eye).

In some embodiments, methods of treatment using the topical ophthalmic compositions described herein result in an at least 3-line improvement from baseline under the condition of photopic, high contrast UDVA. In other embodiments, methods described herein result in an increase in the average letter change from baseline under the condition of photopic, high contrast UDVA.

In certain embodiments, methods of treatment using the topical ophthalmic compositions described herein result in an at least 2-line improvement from baseline under the condition of mesopic, high contrast distance-corrected near visual acuity (DCNVA). As used herein, the term “distance corrected near visual acuity” (DCNVA) refers to a subjects ability to see the details of objects within arm's distance from the body (e.g., at 33-41 cm away from the eye), with the use of vision aids such as eyeglasses or contact lenses that correct for distance vision issues.

In some embodiments, methods of treatment using the topical ophthalmic compositions described herein result in an at least 3-line improvement from baseline under the condition of mesopic, high contrast DCNVA. In other embodiments, methods described herein result in an increase in the average letter change from baseline under the condition of mesopic, high contrast DCNVA. In yet other embodiments, methods described herein result in an at least 3-line improvement from baseline under the condition of photopic, high contrast DCNVA. In additional embodiments, methods described herein result in an at least 2-line improvement from baseline under the condition of photopic, high contrast DCNVA. In further embodiments, methods described herein result in an increase in the average letter change from baseline under the condition of photopic, high contrast DCNVA.

In certain embodiments, methods of treatment using the topical ophthalmic compositions described herein result in an at least 2-line improvement from baseline under the condition of mesopic, high contrast distance-corrected intermediate visual acuity (DCIVA). As used herein, the term “distance-corrected intermediate visual acuity” (DCIVA) may be used to refer to a subject's ability to see the details of objects at intermediate distances with the use of vision aids such as eyeglasses or contact lenses that correct for distance vision issues.

In some embodiments, methods of treatment using the topical ophthalmic compositions described herein result in an at least 3-line improvement from baseline under the condition of mesopic, high contrast DCIVA. In other embodiments, methods described herein result in an increase in the average letter change from baseline under the condition of mesopic, high contrast DCIVA. In yet other embodiments, methods described herein result in an at least 2-line improvement from baseline under the condition of photopic, high contrast DCIVA. In additional embodiments, methods described herein result in an at least 3-line improvement from baseline under the condition of photopic, high contrast DCIVA. In further embodiments, methods described herein result in an increase in the average letter change from baseline under the condition of photopic, high contrast DCIVA.

### EXAMPLES

Employing a fixed small pupil ideal for high light levels may compromise distance vision at low light environments, and a pupil miosis that is ideal for distance vision at low light levels will provide reduced near vision gain at high light levels. Because pupil miotic drugs create pupil constrictions that scale inversely with light level, miotic (e.g., pilocarpine) or antimydriatic (ALPHAGAN P™) drugs may be able to provide the light level dependent pupil miosis required to expand depth of focus while retaining the quality of distance vision over a wide range of environmental lightning.

In the following study, the visual impact of a single drop of 0.1% ALPHAGAN P™ on near vision of early and late presbyopic subjects was examined. The purpose of the study was to determine if this low efficacy miotic produces sufficient pupil miosis to improve near acuity and reading while maintaining distance vision over a range of environmental light levels.

### Example 1: Methods

Subjects

The present study followed the tenets of the Declaration of Helsinki, and the Indiana University Institutional Review Board approved the use of human subjects and consent forms obtained from the subjects after explanation of the nature and possible consequences of the study prior to the beginning the experiment. Thirty presbyopic subjects (age: 40-61 years, 13 subjects with dark iris; 17 subjects with light iris) were divided into two groups: mature presbyopic group (>50 years, 11 subjects) and early presbyopic group (40-50 years, 19 subjects). All subjects have best distance-corrected visual acuity of 20/20 with no ocular or systematic disease.

Pupil size, refraction, visual acuity (VA), near reading performance, and intraocular pressure were monitored for 8 hours at a wide range of light levels following bilateral instillation of single drops of 0.1% ALPHAGAN P™ in the 19 young presbyopes and the 11 mature presbyopes.

Instruments

a. Binocular Open-View Pupilometer:

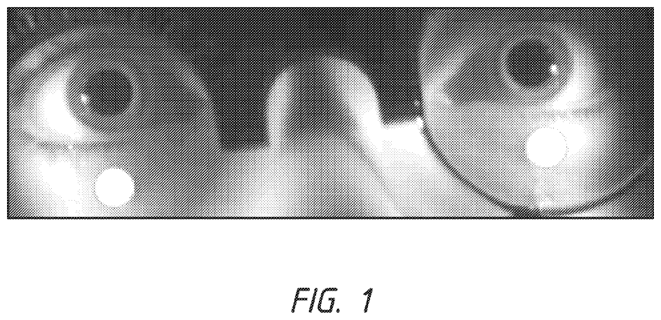

Pupil diameter was measured with a custom built binocular open-field pupilometer mirroring the core design of Chateau, with an infra-red (IR) camera (frame rate: 30 frames per second) that viewed the eye through a hot mirror (Thorlabs.com). Four infrared light emitting diodes (lambda=850 nm) were placed around each eye. Room lights were controlled to generate 0 (0.01 lux with all room light off, which is rounded for convenience to 0 lux throughout the text), 20, 200, and 2000 lux at the distance (5 m) and near (40 cm) acuity charts. The environmental light levels were defined in lux rather than specifying chart luminance (cd/m2), because although the neural response of the visual system to the test stimuli will be directly related to the stimulus luminance, pupil size will be affected by the lighting levels in the whole room. Pupil size was recorded while subjects read test stimuli at distance and near. To account for the varying magnification or minification of the pupil images due to eye distance and trial lens magnification, a 6 mm diameter circular calibration marker was attached to each eye's lower eyelid (FIG. 1). Custom-written MATLAB code was created to report the best fitting circle to the entrance pupil images obtained during acuity and reading testing. A total of 1080 images were analyzed and averaged per subject.

b. Grand Seiko Auto-Refractor:

One concern about the application of ALPHAGAN P™ is that it might indirectly affect accommodation in the early presbyopic population. Therefore, refractive error was measured objectively using a Grand Seiko Auto-refractor while subjects binocularly viewed high contrast distance and near acuity charts placed at 5 m and 40 cm, respectively. Induced accommodation will manifest as a myopic drift during the first hour or two after dosing.

Experimental Design

A single drop of 0.1% ALPHAGAN P™ was vertically dispensed into the inferior cul-de-sac of both the right and left eyes between 7:00 and 8:00 am. Mean weight of a single drop was 0.044 g with a between drop SD of 0.002 g (SD=5% of mean) and a between bottle SD of means from each bottle of 0.0005 g, confirming a previously reported mean weight of 0.046 grams per drop. Each drop of 0.1% ALPHAGAN P™ delivered 0.1%×0.044 g=0.044 mg of brimonidine tartrate, the active ingredient, to the eye (www.alphaganp.com/ALPHAGANP.pdf). Both eyes were refracted at baseline and every 1 hour throughout the day. Pupil sizes were monitored at baseline and then every 15 minutes after instillation for the first hour and then every hour for 8 hours. Plasma levels of ALPHAGAN P™ after a single drop of 0.1% ALPHAGAN P™ have been reported to return to baseline levels before 8 hours, (www.accessdata.fda.gov/drugsatfda_docs/nda/2005/021770s000TOC.cfm).

Distance subjective refraction was measured by an experienced optometrist at baseline using standard high contrast letter optotypes at a photopic light level (500 lux) viewed from 5 m. Both binocular near visual acuity and reading speed were tested at 40 cm with distance correction at three light levels (20, 200, 2000 lux). Near visual acuity was tested with clinical near log MAR acuity charts.

For letter sizes within a factor of two or three of the visual acuity threshold, reading speed varies dramatically with letter size. Therefore, improvements in visual acuity associated with pupil miosis should manifest as improvements in reading speed for small text close to the VA limit. Oral reading speed was captured by digital recordings for a range of text sizes presented in descending size order starting with text that was approximately 2× the measured near visual acuity (which typically produced speeds in excess of 150 words per minute). Reading speed was assessed using high-contrast printed black text on white paper using the methods described by Xu et al (Xu R, Bradley A., Iuread: A New Computer-Based Reading Test, 2015, Ophthalmic Physiol Opt; 35:500-13) with text sizes sampled from −0.27 to 1.24 log MAR and stepped in 0.1 log MAR increments until text was too small to read.

ALPHAGAN P™ is commonly used to lower intraocular pressure of glaucomatous eyes. Therefore, ocular health and intraocular pressure of all subjects were checked prior to drop instillation and at 8 hours following instillation. Also, every hour during testing investigators queried subjects to report any symptoms possibly associated with adverse reactions any time during the experiment. Each subject was asked four questions: (1) Are you experiencing any headache? (2) Are you feeling any eye strain? (3) Are you feeling okay overall? (4) Do you have ANY other ocular symptoms? No subject reported any ocular symptoms.

After 5 minutes of initial dark adaptation, the subject was tested using an ascending order of light levels (0, 20, 200, 2000 lux) to ensure that the retina was progressively light adapting during the experiment. After each room lighting level change, subjects were given 2 minutes to light adapt 35, 36 before pupil and visual performance measures were recorded. Three frames of each eye from within a sample 10 second video stream (300 frames total) were measured for each condition.

### Example 2: Safety Data

As indicated from the FDA medical review and Clinical Pharmacology Biopharmaceutics review data (www.accessdata.fda.gov/drugsatfda_docs/nda/2005/021770s000TOC.cfm), ALPHAGAN P™, in its lower 0.1% formulation has been proven to be a safe and very well tolerated topical ophthalmic drug when applied chronically up to three times daily. In the present study, there were no subjective complaints, no adverse events, and no side effects reported during hourly monitoring for any adverse effects. Also, intraocular pressure remained within the normal range (mean pressure at baseline and 8 hours were 14.3 and 13.1 mmHg, respectively) and no eye experienced a change of >±4 mmHg.

### Example 3: Pupil Size

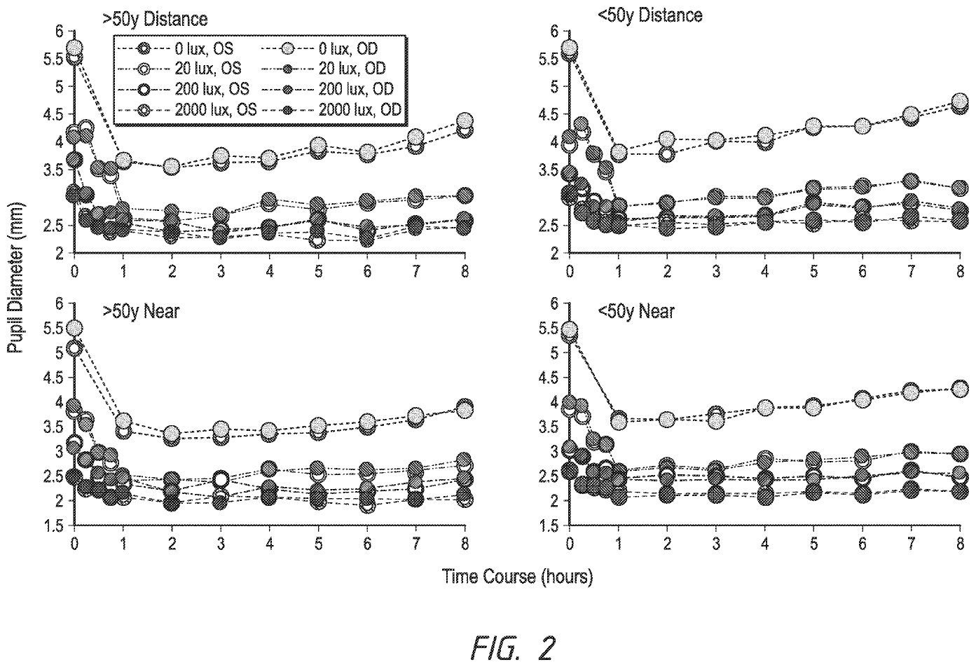

During the 8-hour measurement period, all subjects had nearly identical pupil responses in their right and left eyes (filled and open symbols in FIG. 2) at each light level and viewing distance. Although reduced, the miotic effect of 0.1% ALPHAGAN P™ was observed up to 8 hours after the initial instillation, consistent with the observations of Gerente et al. (Gerente V M, Biondi A C, Barbosa C P, et al., The official journal of the Association for Ocular Pharmacology and Therapeutics; 2007; 23:476-80) with 0.15% ALPHAGAN P™. For example, at baseline, hour 1 and hour 8, the averaged distance pupil sizes at 0 lux were 5.7 mm, 3.7 mm and about 4.5 mm, or equivalently reduced to 63% and 75% of the initial size at 1 hour and after 8 hours. Miotic effects were largest at the lowest light levels. For example, at hour 1 the average distance pupil size reduced by 2 mm (5.7 to 3.7 mm) at 0 lux, but reduced only 0.5 mm from (˜3.0 to 2.5 mm) at 2000 lux. At 1 and 2 hours after dosing, pupils of the mature presbyopic subjects were reduced to 63%, 68%, 77%, and 87% of the pre-drug sizes for light levels of 0, 20, 200, and 2000 lux, respectively. For the young presbyopic subjects, pupils reduced to 67%, 66%, 77%, and 77% their pre-drug sizes for light levels of 0, 20, 200, and 2000 lux, respectively. The near pupil miosis (the difference between the distance and near pupil size at each condition) was present in all natural-pupils (at time 0), but the average near miosis was small, <0.5 mm for all light levels, and became even smaller after instillation of 0.1% ALPHAGAN P™, e.g., <0.3 mm for all light levels at hour 1. Within-subject standard deviations of repeat measures were averaged across all conditions, i.e., time, light level, distance, eye, and the average across all 30 subjects of these within-subject standard deviation estimates of pupil diameter was 0.2 mm. Between subjects variability was quantified by computing the standard deviations of the 30 within-subject means for each condition, which were then averaged across conditions to obtain a single estimate of between subject variability (0.47 mm).

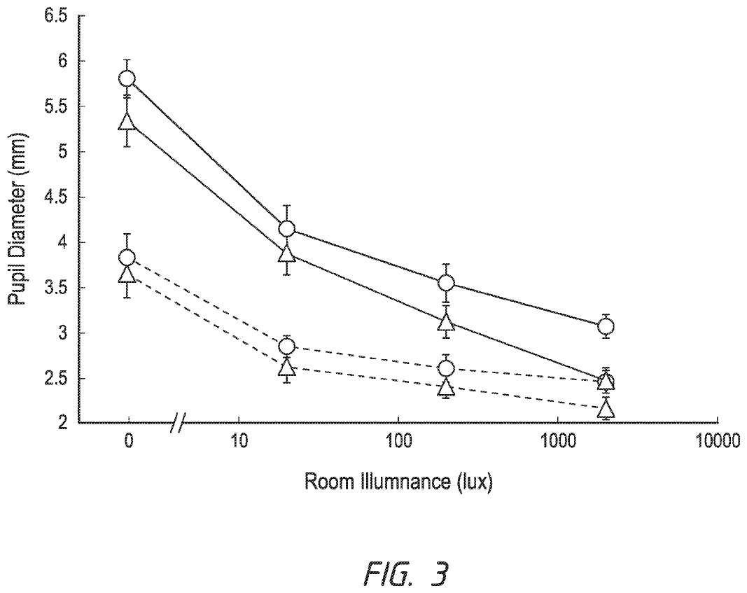

The light level dependency of the miotic effect of 0.1% ALPHAGAN P™ on the >50 year-old eyes is shown in FIG. 3 for both distance (red) and near (blue) viewing by comparing the mean pupil diameters (+/−SEM) at time zero (pre) and 1 hour after instillation (1 h-post). As light levels decrease, the miotic effect of ALPHAGAN P™ increases, but the miotic effect of near viewing (stimulus distance at 40 cm) remains quite stable for all light levels. This plot reveals that, unlike a fixed aperture implant, a miotic retains the ability to dilate at lower light levels, which can compensate for the elevated photon noise occurring at low light levels.

### Example 4: Binocular Vision Acuity Data

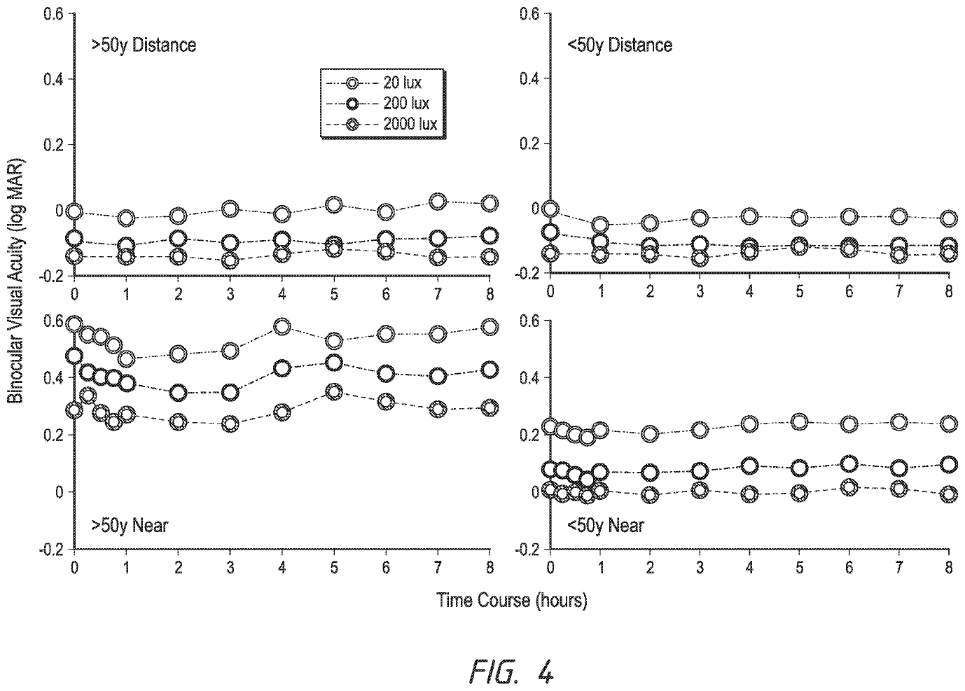

Distance visual acuity improved with increasing light levels (FIG. 4), with an average improvements of 0.07 and 0.14 log MAR as room illuminance was increased from 20 to 200 and then 2000 lux. Pupil miosis did not reduce distance visual acuity at any light level. During the first hour after dosing, younger presbyopes exhibited a small average gain of 0.07 log MAR in distance visual acuity at both 20 lux and 200 lux light levels, which is consistent with pupil miosis (at baseline, mean pupil diameter at 20 lux were 4.1 mm, decreasing to 3.5 mm and 2.7 mm at 200 and 2000 lux). For binocular near acuity (FIG. 4, bottom panels), both age groups showed significantly lower baseline near acuities. Relative to the distance acuities (FIG. 4, top panels), mean near log MAR thresholds increased by 0.5 and 0.17 for the >50 and <50 year old groups. In the >50 year old sample, average visual acuity at hour 1 following instillation improved significantly (Paired two-tailed t-test comparison): mean change in log MAR=−0.15 (p=0.03), −0.07 (p=0.04), and −0.03 (p=0.03), at 20, 200, and 2000 lux, respectively. The smaller improvement at 2000 lux probably reflects the already small natural pupil size (2.7 mm) and small miotic effect of the ALPHAGAN P™ (0.5 mm) at this high light level (FIG. 3). For young presbyopes, there was a small <0.02 log MAR (1 letter) gain in visual acuity at the lowest light level, but no statistically significant change at any light level. Two hours after instillation mean acuities in the >50 group were improved relative to baseline, but only reached 0.05 significance (mean change in Log MAR is −0.11, p=0.008) at 200 lux.

The improved near acuities cannot be attributed to a myopic change in refractive state. At baseline mean (SD) spherical refractive errors measured with the Grand Seiko were −1.0(1.6) and −0.94(1.4) D for the >50 and <50 year-old groups, respectively. One hour after instillation of ALPHAGAN P™, spherical Rx was unchanged in the <50 year-old group, and had become 0.13 D more hyperopic than at baseline in the >50 year-old group.

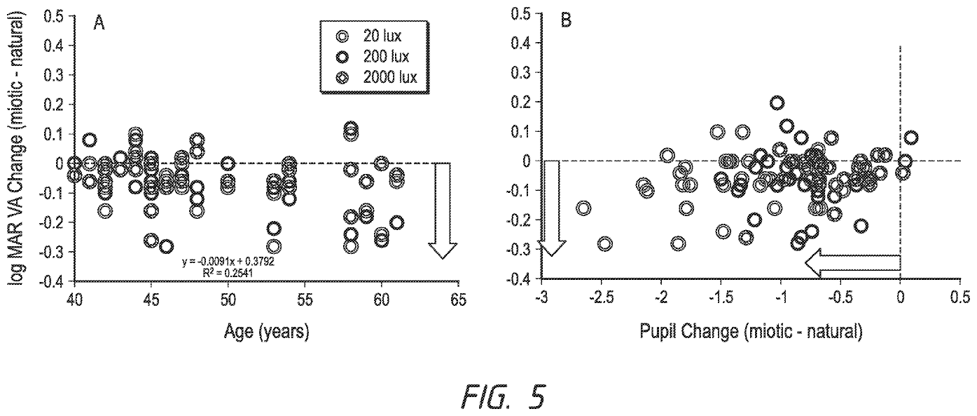

Changes in log MAR acuity following instillation of 0.1% ALPHAGAN P™ showed a significant association with age (correlation coefficient=−0.34, p=0.001, FIG. 5A) which shows that the visual benefits of pupil miosis are most apparent in the older subjects (slope of best fitting line is approximately −0.1 log MAR per decade). The younger sample (40-50 years of age) experienced almost no change in visual acuity with pupil miosis, likely due to residual accommodation, and the reduced impact of pupil miosis when defocus levels are small. The correlation between miosis magnitude and acuity improvements at near is examined in FIG. 5B. At the highest light levels (purple symbols), average pupil miosis was 0.6 mm with an average gain of visual acuity of only 0.04 log MAR. By contrast, at 20 lux (red symbols), pupil miosis varies from 0.5 mm to 2.65 mm, and subjects with increased miotic effect generally achieved the greatest improvement in near acuity (correlation coefficient=0.197, p=0.06).

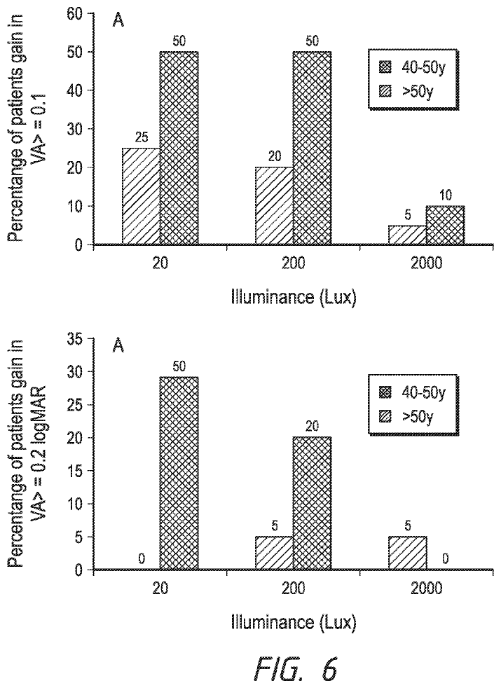

FIG. 6 summarizes the percentage of subjects who showed improvement in acuity at 20, 200, and 2000 lux, for a criterion improvement of 0.1 log MAR (FIG. 5A) or 0.2 log MAR (FIG. 5B). There were 50%, 50%, and 10% of mature presbyopic subjects which showed improvement of at least 0.1 log MAR acuity, whereas only 25%, 20%, and 5% of the early presbyopic subjects gained at least 0.1 log MAR, at 20, 200, and 2000 lux, respectively. These distributions show that the older subjects (>50 years) were most likely to achieve improved near acuity. When raising the criterion to at least a 0.2 log MAR improvement (FIG. 5B), 30%, 20%, and 0% of mature presbyopic subjects reached the criterion, but <5% of the early presbyopic subjects gained >0.2 log MAR. The gain of near visual acuity was lower at the highest light levels because of the smaller miotic effect at these high light levels (also see purple dots in FIG. 4). However, near acuities remained highest at the high light level (FIG. 4).

### Example 5: Reading Performance Data

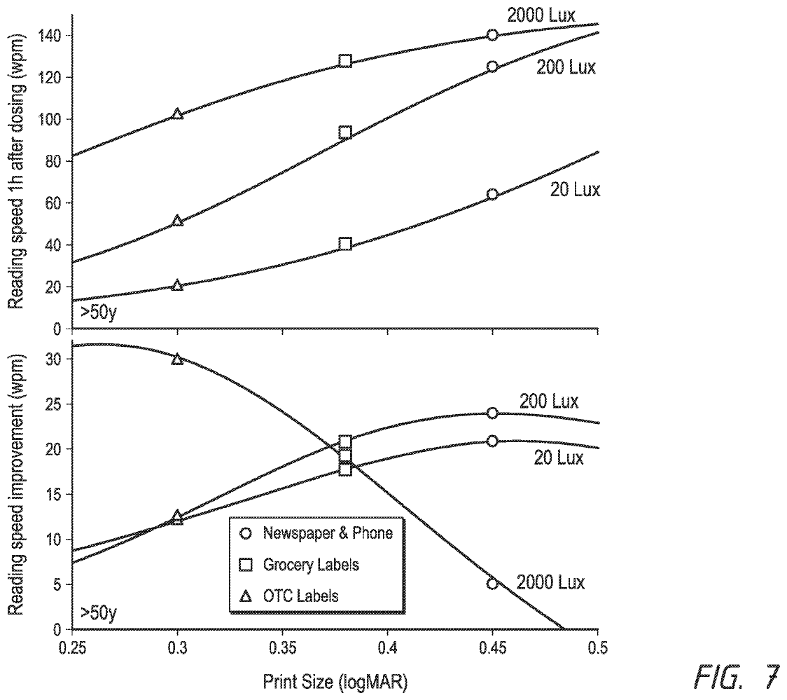

To capture changes in reading speed caused by miosis, reading speed was measured at t=0 and at 1 h-post-ALPHAGAN P™. In the group of mature presbyopes (>50 y), as print size increased, the reading speed also increased for all three light levels (FIG. 7A). The absolute reading speeds are 64, 125, and 140 words per minute for text size commonly seen in US newspaper and cellphone text message (black circles in FIG. 7A), and 40, 94, and 128 words per minute for text size of grocery labels (squares in FIG. 7A), and 21, 52, and 103 words per minute for text size of OTC drugs (triangles in FIG. 7A), at light levels of 20, 200, and 2000 lux respectively. The achieved reading speed with miotics is sufficient (>100 words per minute) to allow mature presbyopes to proficiently read typical newspapers and cellphone message at room light levels (125 words per minute at 200 lux) or outdoors (140 words per minute at 2000 lux). However, only at 2000 lux, the reading speed is sufficient to achieve proficient reading of typical text seen in grocery labels (128 words per minute) and OTC labels (103 words per minute).

The GAIN of reading speed with 0.1% ALPHAGAN P™ is shown in FIG. 7B. At light levels of 20 and 200 lux, the average reading speed in 1 h after dosing is always better than the baseline, achieving largest effect with font sizes between 0.3 and 0.5 log MAR. At 2000 lux, reading speed improvements were observed with smaller text. Across the font size range from 0.25 log MAR to 0.6 log MAR, reading speed on average improved by 17, 19, and 10 words per minute, for light levels of 20 (red curves), 200 (blue curves), and 2000 lux (purple curves), respectively. In addition, reading speed improved 21, 24, and 5 words per minute for text size commonly seen in US newspaper and cellphone text message (black circles in FIG. 7B), and 18, 21, and 19 words per minute for text size of grocery labels (squares in FIG. 7B), and 12, 13, and 30 words per minute for text size of OTC drugs (triangles in FIG. 7B), at light levels of 20, 200, and 2000 lux respectively. A similar improvement was also observed at 2 h after dosing, but no improvements were observed from 3-8 h after dosing. There was no statistically significant improvement observed in the group of young presbyopes (40-50 years) at any time after instillation of ALPHAGAN P™.

The miotic impact of a single dose of 0.1% ALPHAGAN P™ successfully enhanced the near vision in presbyopic patients while retaining high quality distance vision over a wide range of light levels. The miosis achieved significant near reading improvement sufficient to allow mature presbyopes to read typical newspapers and cellphone message proficiently at room light levels (200 lux) or outdoors (2000 lux).

ALPHAGAN P™ did not adversely change distance visual acuity (VA) or refraction. Significant pupil miosis peaked at 1-2 hours after dosing, which expanded the depth of focus of mature presbyopes with the mean improvement of near log MAR VA of −0.15, −0.07, and −0.03, at 20, 200, and 2000 lux, respectively. Compared to the baseline, near reading speed improved 21, 24, and 5 words/minute for text size commonly seen in US newspaper and cellphone text message, and 18, 21, and 19 words/minute for text size of grocery labels, and 12, 13, and 30 words/minute for text size of OTC drugs, at light levels of 20, 200, and 2000 lux respectively. No such improvements in near VA and near reading speed were observed in the young presbyopes with some residual accommodation.

A single drop of low dose (0.1%) ALPHAGAN P™ had no detrimental effects on patient vision (distance acuity) at any light level, produced no myopic shift in refraction, and produced zero reported adverse events at any time during the day. It did not reduce the normal IOP levels in any of the patients. These results emphasize the safety of a single dose of this low dose drug. A single bilateral dose of 0.1% ALPHAGAN P™ was sufficient to generate consistent and highly significant pupil miosis that peaked at 1-2 hours after dosing (pupil diameters reduced to 63% of baseline), with some miosis remaining after 8 hours (pupil diameters 75% of baseline). The induced pupil miosis improved near visual acuity (FIGS. 4-6) and reading speed (FIG. 7) at the lower light levels (20 and 200 lux) in the older (>50) group.

The optimum impact of pupil miosis (maximum gain at near without loss at distance) has been achieved by reducing pupil diameters to about 40% or 30% of their natural size. The low dose 0.1% ALPHAGAN P™ tested in the current study produced a miotic effect was less than optimum (pupils at hour 1 that were between 63% and 80% of pre-dosing). Although reading speed with 0.1% ALPHAGAN P™ is sufficient to allow mature presbyopes to proficiently read typical newspapers and cellphone message at room light level, only at 2000 lux, the reading speed is sufficient to achieve proficient reading of typical text seen in grocery and OTC labels. It has been shown that higher (0.15% and 0.2%) concentrations of ALPHAGAN P™ may generate larger miotic effects than those observed here. This suggests that an increased miotic effect may translate into an increased improvement in near visual acuity as the achieved miosis approaches the optimum observed in the present studies.

The optics of pupil size changes are predictable. Increasing pupil size for a well-focused eye degrades image quality slightly due to aberrations, but increasing pupil size for a defocused eye (e.g., presbyope trying to read at 40 cm) will dramatically lower image quality, resulting in the familiar visual disability that emerging and full distance corrected presbyopes experience at low light levels. However, because neural contrast sensitivity is reduced at low retinal illuminance levels, pupil dilation in low environmental lighting can result in improved vision quality. Therefore, the balance between improved optical quality and reduced neural contrast sensitivity associated with pupil miosis will vary with viewing distance (which determines defocus in presbyopic eyes) and environmental light level (which determines the impact of retinal illuminance on neural contrast sensitivity). Because of these multiple factors, it is not possible to identify a single optimum pupil size for all stimulus distances and all light levels, and therefore a fixed small pupil (e.g., corneal inlay) that is optimal at high light levels will be sub-optimal at low light levels. Although miotic drugs reduce pupil size, the pupil retains its light response and some near miosis (FIG. 3), and thus a pharmacological miotic, unlike a fixed pupil introduced into the visual pathway, has the potential to achieve increased depth of focus by pupil miosis, but retain higher quality distance vision over a wide range of light levels.

Creating a small pupil in the iris plane maintains the wide-angle optical properties so crucial to human vision and mobility, and avoids the field attenuation generated by corneal plane artificial pupils. Also, pupil miosis will not introduce visually disruptive starbursts seen around bright points of light at night that are exacerbated by multifocal optics. Pupil miosis will also avoid the inevitable loss of contrast for distance vision produced by multifocal optics. Also, pupil miosis will not create blur and distortion in the periphery associated with progressive spectacle lenses, and therefore avoid increased risk of falls in older subjects wearing spectacle corrections.

1. A method of treating presbyopia in a subject in need of treatment thereof, comprising administering to at least one eye of the subject a therapeutically effective amount of a topical ophthalmic composition comprising brimonidine or a pharmaceutically acceptable salt thereof.

2. A method for improvement of near vision in a subject with presbyopia in need of treatment thereof, comprising administering to at least one eye of the subject a therapeutically effective amount of a topical ophthalmic composition comprising brimonidine or a pharmaceutically acceptable salt thereof.

3. A method for reducing pupil diameter in a subject with presbyopia in need of treatment thereof, comprising administering to at least one eye of the subject a therapeutically effective amount of a topical ophthalmic composition comprising brimonidine or a pharmaceutically acceptable salt thereof.

4. A method of improving near reading speed in a subject with presbyopia in need of treatment thereof, comprising administering to at least one eye of the subject a therapeutically effective amount of a topical ophthalmic composition comprising brimonidine or a pharmaceutically acceptable salt thereof.

5. The method of claim 1, wherein the brimonidine is present as brimonidine tartrate.

6. The method of claim 5, wherein the brimonidine tartrate is present at a concentration from about 0.01% (w/v) to about 10% (w/v).

7. The method of claim 6, wherein the brimonidine tartrate is present at a concentration from about 0.1% (w/v) to about 0.2% (w/v).

8. The method of claim 7, wherein the brimonidine tartrate is present at a concentration of 0.15% (w/v).

9. The method of claim 7, wherein the brimonidine tartrate is present at a concentration of 0.1% (w/v).

10. The method of claim 1, wherein the brimonidine is present as a free base.

11. The method of claim 1, wherein the topical ophthalmic composition further comprises a viscosity enhancer.

12. The method of claim 11, wherein the viscosity enhancer is selected from the group consisting of carboxymethyl cellulose, hypromellose, hydroxyethyl cellulose, hydroxymethyl cellulose, methylcellulose, methyl cellulose 4000, hydroxypropyl cellulose, hydroxypropylmethyl cellulose, hydroxyl propyl methyl cellulose 2906, carboxypropylmethyl cellulose, hydroxypropylethyl cellulose, and hydroxyethyl cellulose, polyethylene glycol, polyvinyl alcohol, pyrrolidone, polyvinyl pyrrolidone, gellan, carrageenan, alignic acid, carboxyvinyl polymer, glycerol, and acrylic polymers.

13. The method of claim 12, wherein the viscosity enhancer is present at a concentration from about 0.01% (w/v) to about 10% (w/v).

14. The method of claim 12, wherein the viscosity enhancer is carboxymethyl cellulose.

15. The method of claim 14 wherein the carboxymethyl cellulose is present at a concentration of about 0.5% (w/v).

16. The method of claim 1, wherein the topical ophthalmic composition further comprises a buffer.

17. The method of claim 16, wherein the buffer is present at a concentration from about 0.001% (w/v) to about 1% (w/v).

18. The method of claim 16, wherein the buffer is selected from the group consisting of phosphate, borate, borate citrate and lactate buffer.

19. The method of claim 18, wherein the buffer is a borate buffer.

20. The method of claim 19, wherein the borate buffer is sodium borate decahydrate.

21. The method of claim 20, wherein the sodium borate decahydrate is present at a concentration of about 0.045% (w/v).

22. The method of claim 1, wherein the topical ophthalmic composition further comprises one or more osmolality agents.

23. The method of claim 22, wherein the one or more osmolality agents are each present at a concentration from about 0.001% (w/v) to about 20% (w/v).

24. The method of claim 22, wherein the one or more osmolality agents is selected from the group consisting of glycerin, propylene glycol, mannitol, sorbitol, sodium chloride, potassium chloride, and dextrose.

25. The method of claim 1, wherein the topical ophthalmic composition further comprises calcium chloride and magnesium chloride.

26. The method of claim 1, wherein the topical ophthalmic composition further comprises a preservative.

27. The method of claim 26, wherein the preservative is selected from the group consisting of benzalkonium chloride and a stabilized oxychloro complex comprising chlorite, chlorate and chlorine dioxide.

28. The method of claim 27, wherein the preservative is a stabilized oxychloro complex.

29. The method of claim 28, wherein the stabilized oxychloro complex is present at a concentration of about 0.005% (w/v).

30. The method of claim 1, wherein the topical ophthalmic composition further comprises boric acid.

31. The method of claim 1, wherein the topical ophthalmic composition further comprises NaOH and HCl.

32. The method of claim 1, wherein the topical ophthalmic composition has a pH of about 3.0 to about 8.0.

33. The method of claim 32, wherein the topical ophthalmic composition has a pH of about 3.0 to about 5.5.

34. The method of claim 32, wherein the topical ophthalmic composition has a pH of about 6.6 to about 8.0.

35. The method of claim 34, wherein the topical ophthalmic composition has a pH of about 7.4 to about 8.0.

36. The method of claim 35, wherein the topical ophthalmic composition has a pH of 7.7.

37. The method of claim 1, wherein the topical ophthalmic composition is administered at least once daily.

38. The method of claim 37, wherein the topical ophthalmic composition remains effective following administration for a period of time selected from the group consisting of at least about 6 hours, at least about 8 hours, at least about 10 hours, at least about 12 hours, and at least about 24 hours.

39. The method of claim 37, wherein the topical ophthalmic composition is administered twice daily (BID), three times daily (TID), or four times daily (QID).

40. The method of claim 1, wherein the topical ophthalmic composition is administered to a nondominant eye of the subject.

41. The method of claim 1, wherein the topical ophthalmic composition is administered to a dominant eye of the subject.

42. The method of claim 1, wherein the topical ophthalmic composition is administered to both eyes of the subject.

43. The method of claim 1, wherein the method results in an increase of mean near log MAR thresholds of at least about 0.1 from baseline within about 5 minutes following administration of the topical ophthalmic composition.

44. The method of claim 43, wherein the method results in an increase of mean near log MAR thresholds of about 0.5 from baseline within about 5 minutes following administration of the topical ophthalmic composition.

45. The method of claim 1, wherein the method results in a reduction of pupil diameter of at least about 63% of baseline pupil diameter within about 5 minutes following administration of the topical ophthalmic composition.

46. The method of claim 45, wherein the method results in a reduction of pupil diameter of about 80% of baseline pupil diameter within about 5 minutes following administration of the topical ophthalmic composition.

47. The method of claim 1, wherein the method results in an improvement of average reading speed across a font size range from about 0.25 log MAR to about 0.6 log MAR by at least about 17 words per minute from baseline under mesopic conditions within about 5 minutes following administration of the topical ophthalmic composition.

48. The method of claim 1, wherein the method results in an improvement of average reading speed across a font size range from about 0.25 log MAR to about 0.6 log MAR by at least about 10 words per minute from baseline under photopic conditions within about 15 minutes following administration of the topical ophthalmic composition.

49. The method of claim 1, wherein treatment with the topical ophthalmic composition retains distance visual acuity under mesopic conditions compared to baseline.

50. The method of claim 1, wherein treatment with the topical ophthalmic composition retains distance visual acuity under photopic conditions compared to baseline.

51. The method of claim 1, wherein administering the topical ophthalmic composition does not significantly reduce intraocular pressure in the eye of the subject.

52. The method of claim 1, wherein brimonidine is present as the sole active ingredient.

53. The method of claim 1, wherein the subject is greater than or equal to 50 years of age

54. The method of any claim 1, wherein the subject is greater than or equal to 40 years of age.

55. The method of claim 1, wherein the subject is between 40 and 50 years of age.

56. A method of treating presbyopia in a subject in need of treatment thereof, comprising administering to at least one eye of the subject a therapeutically effective amount of a topical ophthalmic composition comprising 0.1% (w/v) brimonidine, 0.5% (w/v) carboxymethyl cellulose, 0.005% (w/v) stabilized oxychloro complex, 0.6% (w/v) boric acid, 0.045% (w/v) sodium borate decahydrate, 0.37% (w/v) sodium chloride, 0.14% (w/v) potassium chloride, 0.006% (w/v) calcium chloride, 0.006% (w/v) magnesium chloride, and NaOH and/or HCl, wherein the topical ophthalmic composition has a pH of 7.7.

57. The method of claim 1, wherein the subject was not previously diagnosed with glaucoma.

58.-62. (canceled)

63. A topical ophthalmic composition for use in the treatment of presbyopia, wherein the topical ophthalmic composition comprises 0.1% (w/v) brimonidine, 0.5% (w/v) carboxymethyl cellulose, 0.005% (w/v) stabilized oxychloro complex, 0.6% (w/v) boric acid, 0.045% (w/v) sodium borate decahydrate, 0.37% (w/v) sodium chloride, 0.14% (w/v) potassium chloride, 0.006% (w/v) calcium chloride, 0.006% (w/v) magnesium chloride, and NaOH and/or HCl, and wherein the topical ophthalmic composition has a pH of 7.7.
